(19) United States
(12) Patent Application Publication
(10) Pub. No.: US 2025/0082499 A1
(43) Pub. Date: Mar. 13, 2025

(54) ACTIVE DRUG DISPENSING OPHTHALMIC DEVICE HAVING A CONTROLLER-RESPONDER ARCHITECTURE

(71) Applicant: Verily Life Sciences LLC, Dallas, TX (US)

(72) Inventors: Christian Gutierrez, South San Francisco, CA (US); Shungneng Lee, South San Francisco, CA (US); Alireza Dastgheib, South San Francisco, CA (US)

(21) Appl. No.: 18/807,692

(22) Filed: Aug. 16, 2024

Related U.S. Application Data
(60) Provisional application No. 63/534,112, filed on Aug. 22, 2023.

(51) Int. Cl. A61F 9/00; A61K 9/00

(52) U.S. Cl. CPC A61F 9/0008; A61K 9/0051; A61K 9/0097

(57) ABSTRACT

An active drug dispensing ophthalmic device can include a plurality of drug reservoirs, each covered by an electrode, and a controller-responder architecture. Electrodissolution of each electrode and the associated drug release can be governed by a controller via a responder. Employing a controller-responder architecture can reduce the number of connections and separate electrical signals required to actively dispense drugs from each of the plurality of drug reservoirs. The controller can be connected to a plurality of responders via a control line bundle and each of the plurality of responders can deliver signals to the electrode(s) covering a portion of a plurality of drug reservoirs. The controller-responder architecture can also employ a composite electrical communication signal to even further decrease the number of electrical connections required from a controller to each of the responders.

### TECHNICAL FIELD

The present disclosure relates to an active drug dispensing ophthalmic device having a plurality of drug reservoirs, and more specifically, to systems and methods for decreasing the number of electrical connections and separate electrical signals required for actively dispensing drugs from each of the plurality of drug reservoirs using a controller-responder architecture.

### BACKGROUND

One way of delivering drugs into the eye is through an active drug dispensing ophthalmic devices. Each active drug dispensing ophthalmic device can include a number of drug reservoirs and an embedded controller to select a drug reservoir at a time to deliver its contents to the eye. As such, each drug reservoir requires at least one unique electrical connection to the controller to control operation, which often includes several additional connections. As the number of drug reservoirs per active drug dispensing ophthalmic device grows, the numerous connections become more difficult to effectively manage and take up much needed space. For example, when more drug reservoirs are added to an active drug dispensing ophthalmic device, a larger controller with additional pads and more electrical connections is required. One way to make room for the additional drug reservoirs, the larger controller, and the additional electrical connections would be by increasing the overall size of the ophthalmic device. However, the size of the ophthalmic devices is limited to what can comfortably fit on and/or in the eye of a subject so the number of drug reservoirs, electrical connections, and size of the controller is limited. Additionally, fabrication challenges increase exponentially with the number of drug reservoirs and electrical connections required and yield can be impacted if drug reservoirs and electrical connections are not perfectly sound. The number of drug reservoirs and doses per active drug dispensing ophthalmic device is thus currently limited and manufacturing is increasingly difficult with the more drug reservoirs included per active drug dispensing ophthalmic device.

### SUMMARY

Active drug dispensing ophthalmic devices can actively deliver a drug to an eye of a user based on a prescribed dosing regimen and/or in response to an automatic control loop. A controller-responder architecture combined with a composite working signal can be used to incorporate a greater number of drug reservoirs in an active drug dispensing ophthalmic device with minimized and simplified electrical connections. For example, with the greater number of drug reservoirs, active drug dispensing ophthalmic devices can be used as longer term wearables (e.g., weekly, bimonthly, or monthly contact lenses), which can deliver more robust combination therapies and/or more continuous therapy for longer times.

In one aspect, the present disclosure includes an ophthalmic device that includes: a controller configured to send an electrical signal to control at least one of a plurality of drug reservoirs; at least one control line bundle configured to transmit the electrical signal to a plurality of responders; the plurality of responders, each connected to the controller via the at least one control line bundle, wherein each of the plurality of responders is configured to receive the electrical signal; and the plurality of drug reservoirs, wherein each of the plurality of drug reservoirs is configured to hold a volume of a drug and is covered by an electrode, wherein each of the plurality of responders is in electrical communication with the electrode of at least one of the plurality of drug reservoirs.

In another aspect, the present disclosure includes a method that includes: determining, by a controller of an ophthalmic device, a selected drug reservoir from a plurality of drug reservoirs from which to release a drug, wherein the ophthalmic device also includes: a plurality of responders connected to the controller via at least one control line bundle, and the plurality of drug reservoirs, wherein each of the plurality of drug reservoirs is configured to hold a volume of the drug and has an opening covered by an electrode, wherein each of the plurality of responders is in electrical connection with the electrode covering at least one of the plurality of drug reservoirs; configuring, by the controller, a working signal including: a power portion configured to power the plurality of responders to a low power mode, and a digital signal portion keyed to a responder in communication with the electrode covering the selected drug reservoir and configured to make the responder in communication with the electrode covering the selected drug reservoir enter a high power state and a remainder of the plurality of responders enter a sleep state; configuring, by the controller, an electrodissolution signal to be sent to the responder in the high power state to trigger release of the volume of the drug stored in the selected drug reservoir; and sending, by the controller, the working signal and then the electrodissolution signal to the plurality of responders via the at least one control line bundle, wherein in response to receiving the working signal and the electrodissolution signal the responder in the high power state is configured to sends the electrodissolution signal to the electrode covering the selected drug reservoir to trigger electrodissolution of the electrode.

In a further aspect, the present disclosure includes a method including: determining, by a controller of an ophthalmic device, a selected drug reservoir from a plurality of drug reservoirs from which to release a drug, wherein the ophthalmic device includes: a plurality of responders connected to the controller via at least one control line bundle, and the plurality of drug reservoirs, wherein each of the plurality of drug reservoirs is configured to hold a volume of the drug and has an opening covered by an electrode, wherein each of the plurality of responders is in electrical connection with the electrode covering at least one of the plurality of drug reservoirs; configuring, by the controller, a working signal including: a power portion configured to power the plurality of responders to a low power mode, a digital signal portion keyed to a responder in communication with the electrode covering the selected drug reservoir and configured to make the responder in communication with the electrode covering the selected drug reservoir enter a high power state and a remainder of the plurality of responders enter a sleep state, and an electrodissolution signal portion to be sent to the responder in the high power state to trigger release of the volume of the drug stored in the selected drug reservoir; and sending, by the controller, the working signal to the plurality of responders via the at least one control line bundle, wherein in response to receiving the working signal the responder in the high power state is configured to sends the electrodissolution signal portion to the electrode covering the selected drug reservoir to trigger electrodissolution of the electrode.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram illustration of an ophthalmic device with a traditional electronic connection system;

FIG. 2 is a block diagram illustration of an ophthalmic device with a controller-responder architecture to decrease the number of electrical connections and separate electrical signals required for actively dispensing drugs from each of the plurality of drug reservoirs;

FIG. 3 is a block diagram illustration of the example ophthalmic device of FIG. 2 with one control line bundle connecting the responders to the controller;

FIG. 4 is an illustration of the example ophthalmic device of FIG. 3;

FIG. 5 is a block diagram illustration of the example ophthalmic device of FIG. 2 with each of the responders connected to the controller via a personal control line bundle;

FIG. 6 is an illustration of the example ophthalmic device of FIG. 5;

FIG. 7 shows illustrations of example control line bundles of FIG. 2 for connecting the controller and the responders;

FIG. 8 is an illustration of the controller-responder architecture of FIG. 2 having a 3-line control bundle;

FIG. 9 is an illustration of an example working signal for the 3-line control bundle system of FIG. 8;

FIG. 10 is an illustration of the controller-responder architecture of FIG. 2 having a 2-line control bundle;

FIG. 11 is an illustration of an example working signal for the 3-line control bundle system of FIG. 10;

FIG. 12 is an illustration of an example control flow diagram for the responder of the device of FIG. 2;

FIG. 13 is an illustration of an example digital code signal portion communication of the device of FIG. 2;

FIG. 14 is an illustration of two way communication between the controller and the responder of the device of FIG. 2;

FIGS. 15-19 are process flow diagrams of example methods for controlling a controller responder system of the device of FIG. 2; and

FIG. 20 shows example diagrams of different connections facilitating electrical communications for different example configurations of ophthalmic devices.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “ophthalmic device” refers to a medical instrument used on or within a portion of a patient's eye for optometry or ophthalmology purposes (e.g., for diagnosis, surgery, vision correction, or the like).

As used herein, an ophthalmic device can be “smart” when it includes one or more components that facilitate one or more active processes for purposes other than traditional lens-based vision correction (e.g., therapeutic release).

As used herein, a smart ophthalmic device can be an “active” ophthalmic device that can deliver a drug from at least one drug reservoir to an eye of a user based on a prescribed dosing regimen, a manual input, and/or in response to an automatic control loop. Unless otherwise stated, as used herein, the term “ophthalmic device” should be understood to mean “active drug dispensing ophthalmic device.”

As used herein, an active drug dispensing ophthalmic device can include a “controller-responder architecture” that controls the electrical communication governing drug delivery. The use of a controller-responder architecture can decrease the number, complexity, and/or length of one or more electrical connections required to activate the same number of electrically responsive components (e.g., an electrode covering a drug reservoir).

As used herein, the term “controller” refers to the “main” or “master” hardware device and/or software program in a controller-responder architecture that initiates and controls transmission of electronical communication to responders in the form of electrical signals.

As used herein, the term “responder” refers to a “subordinate” or “slave” processing device in a controller-responder architecture that receives electrical communication from the controller in the form of electrical signals, processes the electrical signals, and reacts in response to at least one of the processed the electrical signals. A responder can activate one or more connected electrically responsive components during the reaction, for example the responder can send another electrical signal to an electrode to trigger electrodissolution of the electrode.

As used herein, the term “drug reservoir” refers to a storehouse for a volume of a drug. The reservoir can be encapsulated within a body of an ophthalmic device, which may include a hydrogel based material. A portion of the drug reservoir can be open for release of the drug (allowing diffusion of the drug out of the reservoir and into the surrounding hydrogel matrix). The opening may be covered by an electrode to prevent release of the drug. In some instances, the electrode can be used to control the release of the drug from the reservoir via actively controlled electrodissolution.

As used herein, the term “drug” refers to one or more substance (e.g., liquid, solid, or gas) related to the treatment, symptom relief, or palliative care an ocular disorder malady. Drug can include, but is not limited to, a pharmaceutical, a saline component and/or solution, or the like.

As used herein, the term “ocular disorder” refers to a disease, ailment, symptom, or other malady that affects or involves one or more eye, one or more of the parts or regions of the eye, and/or a tissue near the eye. Non-limiting examples of ocular conditions include refractive errors, glaucoma, dry eye, myopia, presbyopia, amblyopia, cataracts, retinopathy, macular degeneration, and the like.

As used herein, the term “electrode” refers to a conductive solid (e.g., including one or more metals, one or more polymers, or the like) that receives/transmits an electrical signal. A non-limiting example of an electrode is a thin-film gold electrode.

As used herein, the term “electrical signal” refers to a signal waveform generated by an electronic means, such as a generator. An electrical signal may be a voltage signal, a current signal, or the like. The electrical signal can include digital and/or analog information. An electrical signal can include one or more communication parts or portions depending on the waveform of the electrical signal over time.

As used herein, the term “waveform” refers to the graphic representation of the shape of a wave that indicates the characteristics, such as frequency and amplitude, of the electrical signal. The waveform can include one or more parts that can be translated as digital and/or analog information by a controller and/or responder.

As used herein, the term “electrodissolution” refers to a process for dissolving a solute using an electrical catalyst. In one non-limiting example, application of an electrical signal to a solid metal can cause the solid metal to dissolve into separate molecules, such as when an electrical signal with proper parameters is delivered to an electrode covering an opening of a reservoir such that the electrode electrodissolves and releases the contents of the reservoir.

As used herein, the terms “patient,” “subject,” “user,” and the like can be used interchangeably and can refer to an animal (e.g., a human) that can wear and/or use an ophthalmic device.

### II. Overview

An active drug dispensing ophthalmic device can treat an ophthalmic disorder by delivering a drug to the eye via one or more drug reservoirs each holding a drug and connected to a controller of the ophthalmic device. As more drug reservoirs are required in the ophthalmic devices (e.g., longer use time, more drug(s), more dosages per time period, or the like) more connections are necessary between the drug reservoirs and the controller and the controller size increases to make room for each of the connections. However, there is an upper size limit of an active drug dispensing ophthalmic device that can comfortably fit in and/or on the eye without disrupting vision. Thus, ophthalmic devices are limited in size. With traditional electronics configurations, as more drug reservoirs are added more electrical connections are required taking up valuable space and increasing the complexity of manufacturing. Each drug reservoir requires an electrical connection to the controller to control operation, and a dedicated ground and/or reference connection. If the ophthalmic device is designed to administer a hundred dosages, then there needs to be at least as many wires going from the controller to each of the drug reservoirs. For current active drug dispensing ophthalmic devices at least four dedicated electrical connections are needed for power, clock, selection, and electrodissolution signals per drug reservoir and the dedicated ground and/or reference line. Current challenges include incorporating a large number of drug reservoirs into an ophthalmic device to create an active drug dispensing ophthalmic device with a simplified manufacturing process.

Described herein is a way to simplify the electrical connections using a controller-responder architecture that can be implemented in an active drug delivery ophthalmic device. A control line bundle travels between the controller and at least one responder and the responder has connections to the electrodes covering drug delivery reservoirs. The control line bundle can comprise a ground line and a signaling line, wherein the working signal comprises a power signal portion, at least one digital code signal portion, and an electrodissolution signal. Alternatively, the control line bundle can include a ground line, a signaling line, and an electrodissolution line, where the working signal comprises a power signal portion and at least one digital code signal portion. The number of electrical connections per drug reservoir can be minimized and controller size (e.g., pad count) can also be minimized with the implementation of the controller-responder architecture, reducing the number of electrical routings of a smart ophthalmic device for drug delivery, thus enhancing the lens fit/comfort and manufacturability. Instead of four or five dedicated connections from the controller to each electrode covering a drug reservoir, the controller can connect to a number of responders, each with only two or three dedicated connections and then each responder can be in communication with a number of electrodes.

### III. System

An ophthalmic device (also referred to as an active drug dispensing ophthalmic device) can deliver one or more drugs to a patient's eye to treat an ophthalmic disorder. The ophthalmic device can be, for example, a contact lens that can be positioned on the surface of an eye, an eye implant, or any other ophthalmic device that can be in contact with at least a portion of the eye of a patient. The ophthalmic device can have a body that can encapsulate all the components described herein. The body of the ophthalmic device can be at least partially made of a hydrogel-based material (e.g., a soft contact lens material) such as a hydrogel or a silicone-hydrogel material, including, but not limited to, all hydrogel and silicone-hydrogel materials. Other materials that may be used in a soft contact lens are also included as or within a hydrogel-based material. A hydrogel can include any crosslinked hydrophilic polymer that does not dissolve in water and is generally highly absorbent yet maintains a well-defined structure.

Such an ophthalmic device can store the one or more drugs in at least one drug reservoir encapsulated within the body of the ophthalmic device. Each of the drug reservoirs can have an interior volume configured to hold a volume of at least one drug. The drug reservoirs can be made of photo-patternable polymers such as an epoxy-based negative photoresist material (SU-8), a positive photoresist material (AZ 1500), a cyclic olefin copolymer (COC), a cyclic olefin polymer (COP), or other thermoplastic polymers such as liquid crystal polymer (LCP), Parylene, Polyimide, polypropylene, polycarbonate, Ultem or Nylon. The at least one drug can be stored in the drug reservoirs in a solid state, a liquid state, or a gaseous state. Each drug reservoir can store one drug or multiple drugs, in any combination. For example, a first drug reservoir can hold a first drug and a second drug reservoir can hold a second drug, where both drugs are different. In another example the first drug reservoir can hold both a first drug and a second drug and the second drug reservoir can also hold both the first drug and the second drug.

Each of the drug reservoirs can have an opening to the interior volume covered by an electrode. The electrodes can be metal electrodes. For example, the electrodes can be thin film gold electrodes. The gold can be thin enough to facilitate the electrodissolution (and in some instances, the length of time to achieve electrodissolution can be based on the thickness of the gold). Other non-limiting examples of electrochemically active metals include silver, platinum, and copper. The electrodes can be triggered to electrodissolve when a specific electrical signal or portion of an electrical signal is applied via the connection with the associated responder. The electrodes can be electrodissolved (e.g., one at a time) upon receiving an electrical signal from a controller, thereby releasing the drug into the patient's eye. The body of the ophthalmic device can be designed such that once a drug is released from a drug reservoir (e.g., by the electrodissolution of the electrode covering the reservoir) the drug can diffuse into/onto the eye. The rate of diffusion of the drug into/onto the eye can be known.

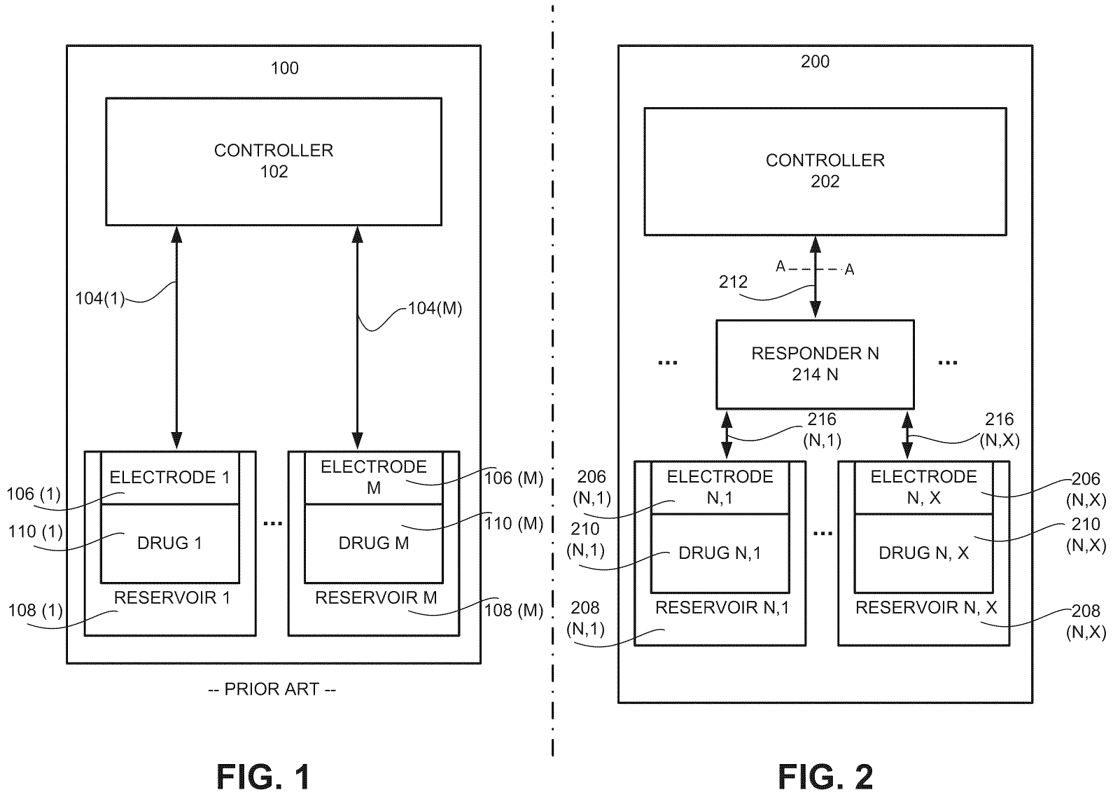

One way to set up the connections between the controller and the electrodes covering each of the drug reservoirs is via a controller-reservoir architecture (employed by the traditional ophthalmic device as shown in FIG. 1). In the traditional controller-reservoir architecture, the controller 102 can be directly connected to one or more drug reservoirs (a plurality is shown, Reservoir 1-Reservoir M, where M is an integer greater than 1) 108 (1)-(M). Each of the drug reservoirs (Reservoir 1-Reservoir M) 108 (1)-(M) can have an interior volume that can hold a volume of a drug (Drug 1-Drug M) 110 (1)-(M) and an opening of each of the drug reservoirs can be covered by an electrode 106 (electrode 1-electrode M) (1)-(M). A controller 102 can be connected to each electrode 106 (1)-(M) with separate wired connections 104 (1)-(M).

Each of the wired connections 104 (1)-(M) can include, for example, one or more dedicated lines for transmitting an electrodissolution signal, a ground and/or counter signal, and a reference signal. In some instance a common ground and/or counter signal line and a common reference signal line can be shared between all drug reservoirs 108 (1)-(M). Selection and timing decisions can be made internally within the controller 102 to determine which wired connection 104 (1)-(M) to send at least the electrodissolution signal through to the electrode 106 (electrode 1-electrode M) (1)-(M). Each line of the wired connections 104 (1)-(M) requires a separate pad on the controller 102 and must be electrically isolated from each of the other lines to remove signal contamination and/or yield loss. With a controller-reservoir architecture, adding additional drug reservoirs (e.g., 108 (M+1)) to an ophthalmic device 100 also includes adding substantially more, and more complex, wired electrical connections (e.g., 104 (M+1) and increasing the size of components, such as the controller 102, while the overall size of the ophthalmic device 100 is limited to what can comfortably fit on and/or in the eye and not disrupt the user's vision. At a certain point, the number of drug reservoirs, connections, and controller size cannot be increased in the available space without cluttering the ophthalmic device 100 and negatively impacting functionality.

Another way to set up the connections between the controller and the electrodes is via a controller-responder architecture (employed by the ophthalmic device as shown in FIG. 2). The controller-responder architecture can save space and improve functionality of the ophthalmic device for greater numbers of drug reservoirs. The controller-responder architecture minimizes the number, complexity, and/or length of electrical connections needed. The ophthalmic device 200 can include a controller 202 connected to a plurality of responders (only one responder, Responder N 214 N, shown for ease of illustration, where N is an integer greater than or equal to 1 and less than or equal to M) with at least one control line bundle 212. Optionally, the ophthalmic device can be made of modular elements connected together and encapsulated within the body material. For example, the controller and the at least one control line bundle 212 can be embodied in an active electronics module and the plurality of responders (Responder N) 214 N and the plurality of drug reservoirs (Reservoir N,1-N,X) 208 (N,1)-(N,X) can be embodied in at least one drug container module. In another example, the controller 202, the at least one control line bundle 212 and the plurality of responders (Responder N) 214 N can be embodied in at least one active electronics module and the plurality of drug reservoirs (Reservoir N,1-N,X) 208 (N,1)-(N,X) and the connections from the plurality of drug reservoirs to the plurality of responders can be embodied in at least one drug container module.

The control line bundle 212 can include, for example, the functionality of four or five dedicated lines for transmitting a power signal, a digital clock signal, a digital selection signal, an electrodissolution signal, and a ground and/or reference signal. Each of the plurality of responders (e.g., Responder N 214 N) can be connected to an electrode (e.g., Electrode N,1-NX) 206 (N,1)-(N,X) where X is a integer greater than or equal to 1 and less than or equal to M) covering at least one drug reservoir (e.g., Reservoir N,1-Reservoir N,X,) with a simpler connection 216 (N, 1)-216 (N, X) than the control line bundle 212 N (e.g., the simpler connection can only transmits the electrodissolution triggering signal from the responder to one of the electrodes).

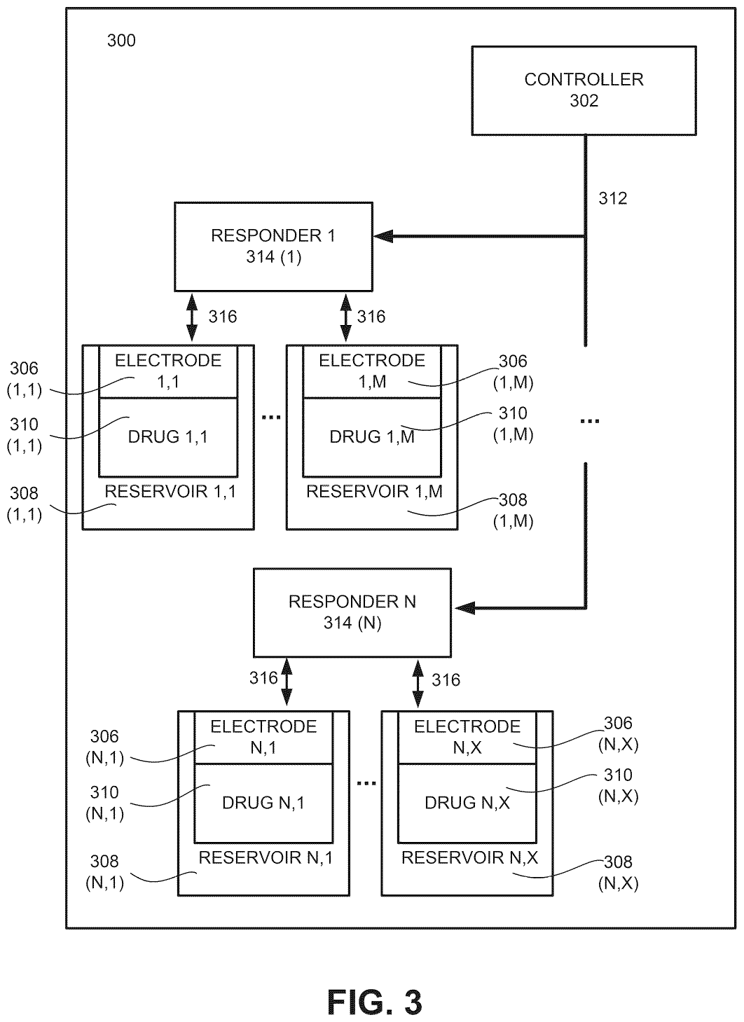

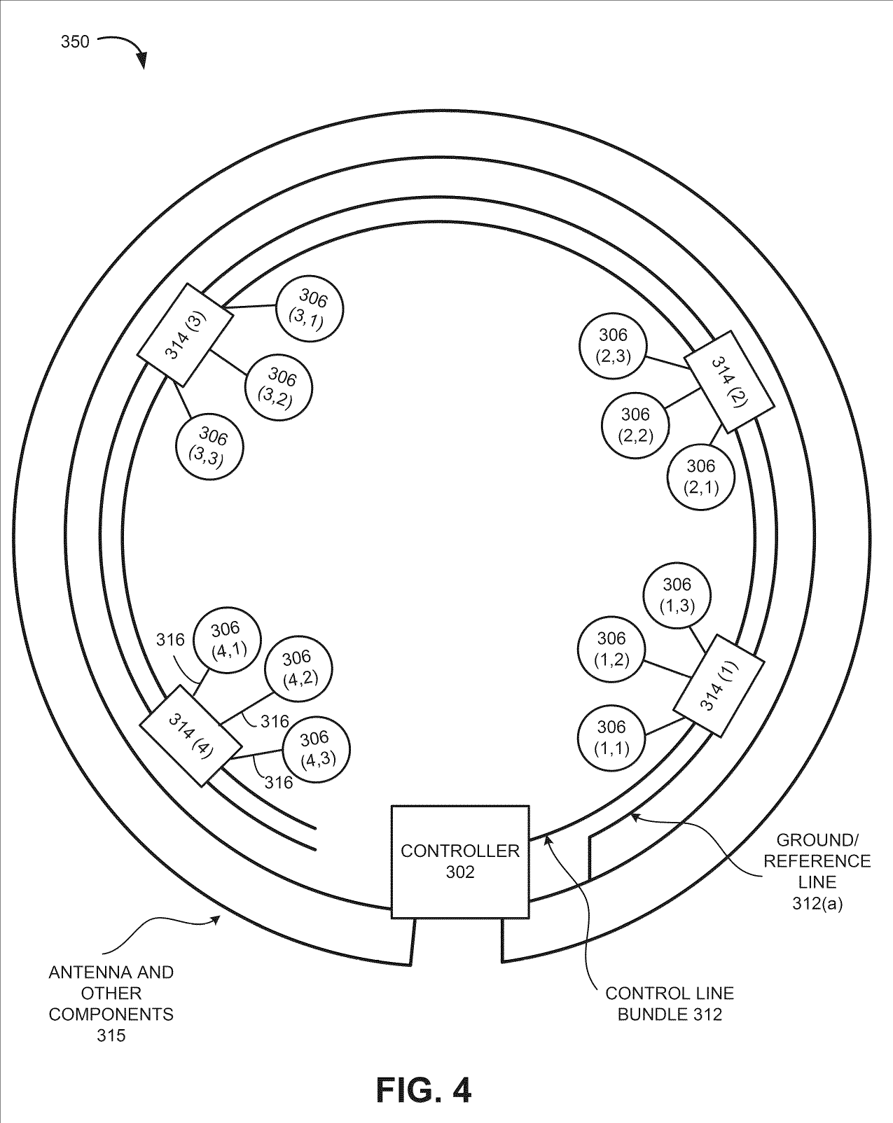

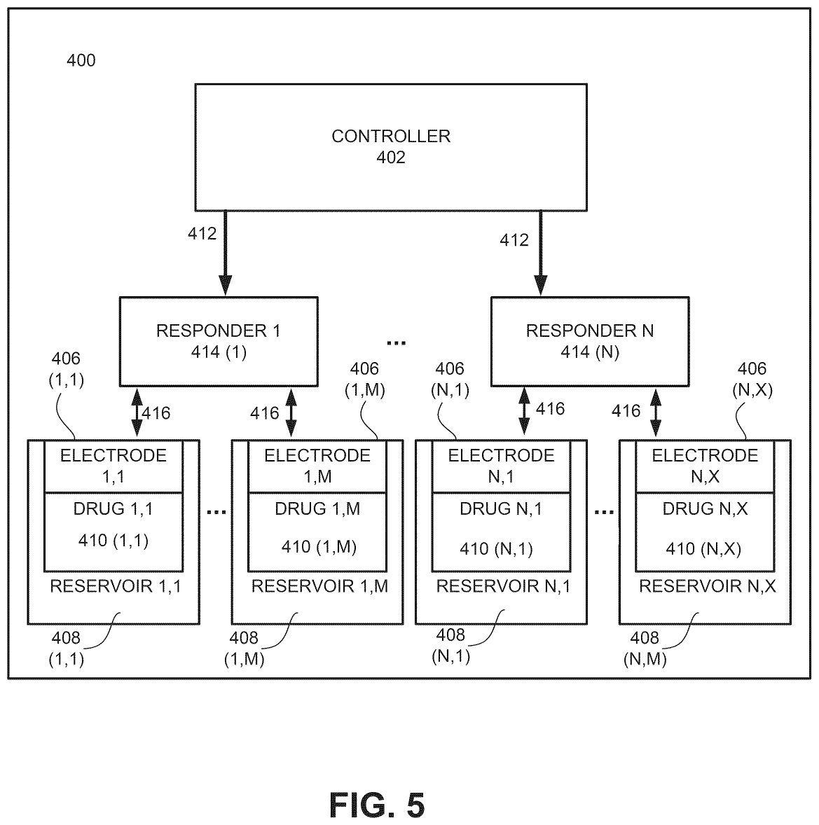

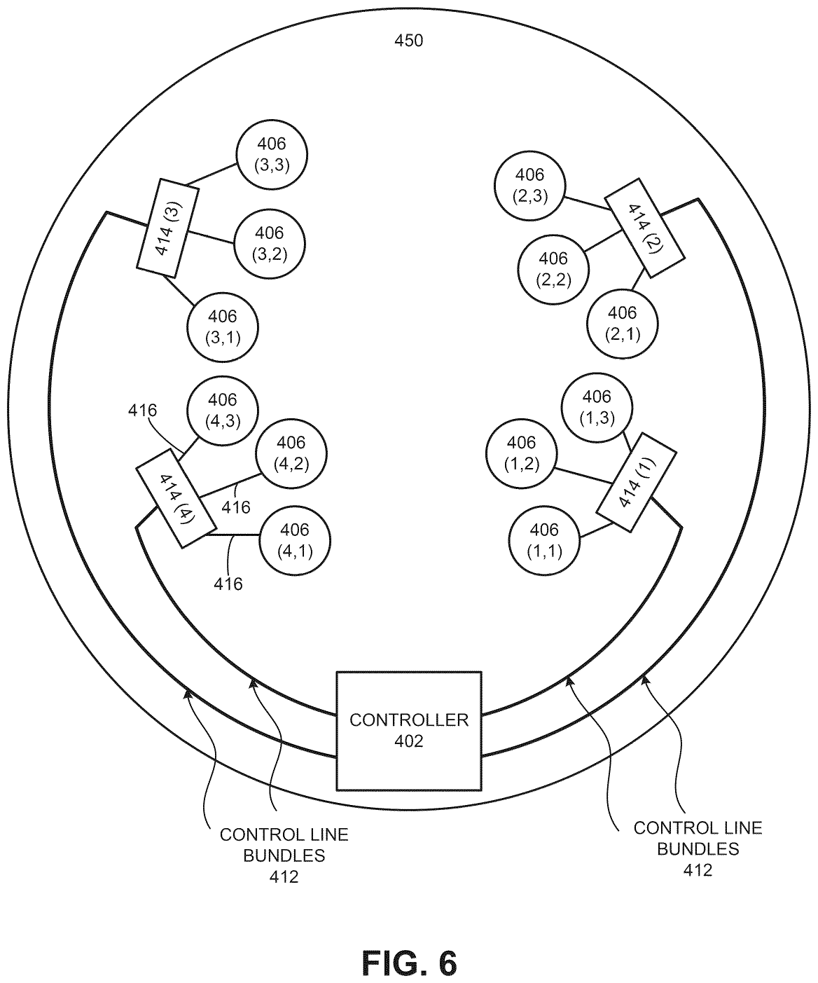

The controller 202 can send an electrical signal (e.g., the unique compound signal) ultimately to control at least one of the plurality of drug reservoirs (Reservoir N,1-Reservoir N,X) 208 (N,1)-(N,X). The electrical signal can be transmitted via the control line bundle 212 to the responder N (or a plurality of responders 1-N). It should be understood that one control line bundle 212 can communicate with (send an electrical signal to) a single responder in some instances (e.g., shown in FIGS. 5-6). However, in other instances, one control line bundle 212 can communicate with (send an electrical signal to) a plurality of responders (including a subset of the responders or every one of the responders) (e.g., shown in FIGS. 3-4). The control line bundle(s) 212 can be electrically isolated from each other and internally (e.g., each line of the control line bundle(s) can be electrically isolated from the other within the bundle).

The controller 202 can store and execute instructions (e.g., computer executable instructions) related to the running and control of the ophthalmic device 200 and the delivery of drug(s) 210 into/onto the eye. In some instances, the controller 202 can include a signal generator that can generate electrical signals that can be sent to the responders (Responder N) 214 N. The electrical signals, described in more detail below, can include at least one waveform (e.g., voltage waveform) based on at least one set of parameters (e.g., magnitude, timing, shape, pulsing, etc.) that can be determined by the controller based on an input (e.g., from a sensor, from a responder, an internal clock component, etc.) and/or manually input. The controller 202 can also store and/or execute additional instructions, data, and information. For example, the controller 202 can be implemented as a type of processor. For example, the controller 202 can be and/or include a microprocessor. The processor can be, for example, embedded within one or more application specific integrated circuits (ASICs), microprocessors, other units designed to perform the functions of a processor, or the like. The controller 202 can have a memory coupled to the processor (e.g., the functionality may be implemented by separate chips). However, in some instances the memory and the processor can be implemented together (e.g., embodied within the same chip) (e.g., a microcontroller device). Optionally, the controller 202 can be in wireless communication with an external device comprising at least one of a display (e.g., a video screen), a memory and a processor, and an input device (e.g., a keyboard, touch screen, and/or a mouse). The controller 202 can also be configured to receive feedback signals from at least one of the plurality of responders (Responder N) 214 N through the at least one control line bundle 212.

With the controller-responder architecture of ophthalmic device 200 each of the plurality of responders (Responder N) 214 N can be connected (e.g., in electrical communication) to the controller via at least one control line bundle 212. One or more separate ground and/or reference lines (not shown) may also connect from the controller 202 with each of the plurality of responders (Responder N) 214 N. Each of the responders (Responder N) 214 N can be in communication, one way or two way, with the controller 202. Each of the responders (Responder N) 214 N can store and/or execute instructions, data, and information. Each of the responders (Responder N) 214 N can be subordinate to the controller 202, e.g., only reacting to signals received from the controller. As an example, each of the responders (Responder N) 214 N can be implemented as a type of processor or at least a portion of processor. For example, each of the responders (Responder N) 214 N can be a microprocessor or at least a portion of a microprocessor. The processor can be, for example, embedded within one or more application specific integrated circuits (ASICs), microprocessors, other units designed to perform the functions of a processor, or the like. Each of the responders (Responder N) 214 N can have a memory coupled to the processor (e.g., the functionality may be implemented by separate chips). However, in some instances the memory and the processor can be implemented together (e.g., embodied within the same chip) (e.g., a microcontroller device). Additionally, each of the responders (Responder N) 214 N, or each of the drug reservoirs (Reservoir N,1-N,X) 208 (N,1)-(N,X) connected to the responders, can include, for example at least one sensor configured to detect at least one characteristic of the electrodissolution and/or a drug stored in the at least one of the plurality of drug reservoirs.

As noted, the ophthalmic device 200 with the controller-responder architecture can have at least two configurations: first, as shown in FIGS. 3 and 4, ophthalmic device 300 can have a single control line bundle 312 connecting the controller 302 with each of the responders 314 (1)-(N) and second, as shown in FIGS. 5 and 6, ophthalmic device 400 can have one control line bundle 412 separately connecting the controller 402 to each of the responders 414 (1)-(N), such that a plurality of control line bundles connect to the controller.

Referring now to FIG. 3, the ophthalmic device 300 can include a controller 302 connected to each of the responders (Responder 1-Responder N) 314 (1)-(N) with a single control line bundle 312. The controller 302 can send an electrical signal (e.g., the unique compound signal) to control at least one of the plurality of drug reservoirs (Reservoir 1,1-Reservoir N,X) 308 (1,1)-(N,X) over the control line bundle 312. The control line bundle 312 can transmit the electrical signal from the controller 302 to the plurality of responders (Responder 1-Responder N) 314 (1)-(N). Each of the plurality of responders (Responder 1-Responder N) 314 (1)-(N) can receive the electrical signal from the controller and can react to one or more portions of the electrical signal. For example, the electrical signal can indicate that a selected drug reservoir from the plurality of drug reservoirs (Reservoir 1,1-N,X) 308 (1,1)-(N,X) should release the drug (Drug 1,1-Drug N,X) 310 (1,1,)-(N,X) stored therein. The electrical signal can include, for example a digital code linked to an associated unique ID of a responder and the associated drug reservoir that is selected (as described in more detail below). Each of the plurality of responders (Responder 1-Responder N) 314 (1)-(N) can be associated with a unique ID (e.g., burned into a memory of the responder) and can be connected (by connection(s) 316) to at least one (preferably a plurality of) drug reservoir (Reservoir 1,1-N,X) 308 (1,1)-(N,X) by the electrode (Electrode 1,1-N,X) 306 (N,1)-(N,X) covering the opening of each of the drug reservoirs (where each reservoir may also have a unique ID). After the responders (Responder 1-Responder N) 314 (1)-(N) receives the electrical signal indicated the selected drug reservoir (Reservoir 1,1-N,X) 308 (1,1)-(N,X) that should release the drug (Drug 1,1-Drug N,M) 310 (1,1,)-(N,X), then the responder associated with the selected drug reservoir can send another electrical signal to the electrode (Electrode 1,1-N,X) 306 (N,1)-(N,X) covering the selected drug reservoir to begin electrodissolution of the electrode and release of the drug.

FIG. 4 shows an example ophthalmic device that is a contact lens 350 (similar to ophthalmic device 300) with four responders 314 (1)-(4) and twelve total drug reservoirs (not visible in this view) each covered by an electrode 306 (1,1)-(4, 3). It should be understood this is only an example and any number of responders and drug reservoirs, as well as other ophthalmic device configurations, are covered. The controller 302 is connected to each of the responders 314 (1)-(4) by a control line bundle 312. A ground and/or reference line 312 also connects each of the responders 314 (1)-(4). In some instances, the ground and/or reference line 312(a) can be a part of the control line bundle 312. Each of the responders 314 (1)-(4) is connected by connectors 316 to three electrodes 306 (Y,1)-(Y,3) where Y is the responder number. A communications antenna and other components 315 such as circuity, a power source, substrates, etc. are also shown.

As an example, the contact lens 350 of FIG. 4 can be a modularized assembly with at least one drug container module and at least one active electronics module. The at least one drug container module can include a substrate (not shown), at least one responder (e.g., responder 314 (1)), associated drug reservoirs covered by electrodes 306 (1,1)-(1,3), and an ability to bond to the two discontinuous concentric rings coming from the controller 302 (shown as the control line bundle and the reference and/or ground line 312). The at least one active electronics module can include the controller 302, the control line bundle 312, and the reference and/or ground line 312. In another example, the plurality of responders 314 (1)-(4) can be part of an active electronics module and the interconnects can attach connections 316 to the responders.

Referring now to FIG. 5, illustrated is an example ophthalmic device 400 that can include a controller 402, a plurality of control line bundles 412, and a plurality of responders (Responder 1-Responder N) 414 (1)-(N), where one of the plurality of control line bundles is paired with one of the plurality of responders. The controller 402 can send an electrical signal (e.g., the unique compound signal) to control at least one of the plurality of drug reservoirs (Reservoir 1,1-Reservoir N,X) 408 (1,1)-(N,X) over one or more of the control line bundles 312. One control line bundle 412 can transmit the electrical signal from the controller 302 to one of the plurality of responders (Responder 1-Responder N) 414 (1)-(N). Each of the plurality of responders (Responder 1-Responder N) 414 (1)-(N) can receive the electrical signal from the controller and can react to one or more portions of the electrical signal. For example, the electrical signal can indicate that a selected drug reservoir from the plurality of drug reservoirs (Reservoir 1,1-N,X) 408 (1,1)-(N,X) should release the drug (Drug 1,1-Drug N,X) 410 (1,1,)-(N,X) stored therein. Each of the plurality of responders (Responder 1-Responder N) 414 (1)-(N) can be connected (by connection(s) 416) to at least one (preferably a plurality of) drug reservoirs (Reservoir 1,1-N,X) 408 (1,1)-(N,X) by the electrode (Electrode 1,1-N,X) 406 (N,1)-(N,X) covering the opening of each of the drug reservoirs. After the responders (Responder 1-Responder N) 314 (1)-(N) receives the electrical signal indicated the selected drug reservoir (Reservoir 1,1-N,X) 408 (1,1)-(N,X) that should release the drug (Drug 1,1-Drug N,X) 410 (1,1,)-(N,X), then the responder associated with the selected drug reservoir can send another electrical signal to the electrode (Electrode 1,1-N,X) 406 (N,1)-(N,X) covering the selected drug reservoir to begin electrodissolution of the electrode and release of the drug.

FIG. 6 shows an example contact lens 450 (similar to ophthalmic device 400) with four responders 414 (1)-(4) and twelve total drug reservoirs each covered by an electrode 406 (1,1)-(4, 3). It should be understood this is only an example and other numbers of responders and drug reservoirs and other ophthalmic device configurations are covered. In example ophthalmic device 400 each of the responders 414 (1)-(4) are separately connected to the controller 401 by dedicated control line bundles 412. Additional circuitry and/or structural components are not shown but are contemplated, including, but not limited to, substrates, additional circuitry, wireless transmitters, antennas, power source(s), etc.

As an example, the contact lens 450 of FIG. 6 can be a modularized assembly with at least one drug container module and at least one active electronics module. The at least one drug container module can include a substrate (not shown), at least one responder (e.g., responder 414 (1)), associated drug reservoirs covered by electrodes 406 (1,1)-(1,3), and an ability to bond to at least one of the control line bundles 412 from the controller 402. The at least one active electronics module can include the controller 402 and the control line bundles 412 (which can include the reference and/or ground line). An interconnect can connect the control line bundles 412 of the active electronics module to each of the plurality of responders 414 (1)-(4) in the at least one active electronics module. In another example the plurality of responders 414 (1)-(4) can be part of an active electronics module and the interconnects can be attached through connections 416 to the responders.

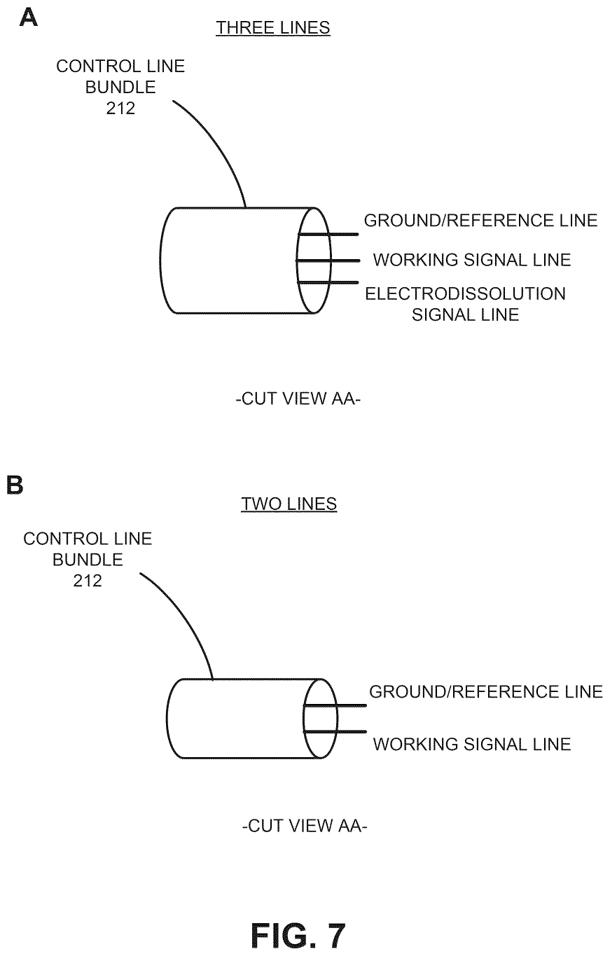

Referring now two FIG. 7, two different control line bundle 212 configurations are shown with reference to cut line A-A of FIG. 2. Both control line bundle 212 configurations correspond with unique compound signaling methods that allow for fewer lines to be used to transmit the same amount of information as traditional five-line controller-responder architecture connections (e.g., power line, digital code line one (e.g., clock), digital code line two (e.g., select), analog signal line, and reference/ground signal line). FIG. 7, element A shows a control line bundle 212 including three lines: a ground and/or reference signal line (which may be part of the control line bundle (as shown) or separate from the control line bundle (not shown)), a working signal line, and an electrodissolution signal line. Such a control line bundle 212 can be received by responders having three pins. FIG. 7, element B shows a control line bundle 212 including two lines: a ground and/or reference signal line (which may be part of the control line bundle (as shown) or separate from the control line bundle (not shown)) and a working signal line. Such a control line bundle 212 can be received by a responders having two pins. By decreasing the number of lines within a control line bundle significant space savings can be realized because smaller controllers per number of drug reservoirs can be used, less and/or shorter lines are needed to transmit the same amount of information per drug reservoir, and smaller responders can be used (with less pins).

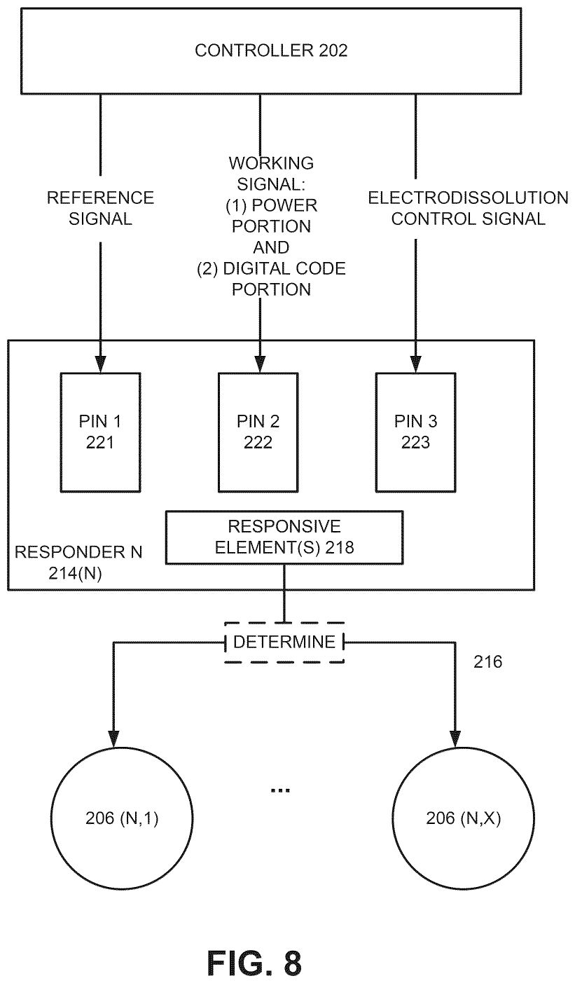

FIG. 8 shows an example diagram of the controller-responder architecture with the three-line control line bundle of FIG. 7, element A. The controller-responder architecture with the three-line control line bundle can include a controller 202 and a plurality of responders (one responder represented as Responder N 214 (N)) having three pins. In this example, the electrical signal transmitted from the controller 202 to each of the plurality of responders can include at least a ground and/or reference signal, a working signal and an electrodissolution control signal. It should be understood that the pin numbering is for ease of description and any pin could connect with any line. Pin 1 221 can connect the reference signal (and/or ground signal) line from the controller 202 to the responder(s). The ground and/or reference signal line can send the ground and/or reference signal to each of the plurality of responders. Pin 2 222 can connect a signaling line from the controller 202 to the responder(s) to send the working signal from the controller to the responder(s). The signaling line can transmit the working signal to each of the plurality of responders. The working signal is the compound signal. In the three-line control line bundle example the working signal can include both a power signal portion for powering the responder(s) and at least one digital code portion overlayed over the power signal to indicate at least the timing and selection of a responder and an associated drug reservoir to release a drug. For example, the at least one digital code portion can select at least one the plurality of responders (here, 214 (N)) and then select at least one electrode 206 (N,1)-(N,X) connected to the selected at least one of the plurality of responders to be electrodissolved.

Pin 3 can connect the electrodissolution control signal line from the controller 202 to the responder(s) (represented here as responder 214 (N)). The electrodissolution control line can send the electrodissolution (control) signal to the selected at least one electrode 206 (N,1)-(N,X) through the selected at least one of the plurality of responders (e.g., 214 (N)) to trigger electrodissolution of the selected at least one electrode. The electrodissolution control signal can be, for example, a voltage signal to be at least partially sent through the responder 214 (N) to the electrode 206 (N,1)-(N,X) covering the selected drug reservoir. The electrodissolution signal can be a voltage waveform configured to trigger electrodissolution of an electrode. For example, the voltage signal can be an analog ramp starting at 2 V and ramping down to 0 V (although other waveforms are possible). The responder(s) can also include at least one responsive element 218 such as a chip, a memory, or a processor that can react to receiving at least a part of an electrical signal from the controller 202. As shown in FIG. 8, the reaction of the responder 214 (N) can be to determine which of the electrodes 206 (N,1)-(N,X) to electrodissolve to release the drug from the selected drug reservoir. When the determination is made (based on the working signal) at least a portion of the electrodissolution control signal can be sent from the responder 214 (N) to the determined electrode of electrodes 206 (N,1)-(N,X).

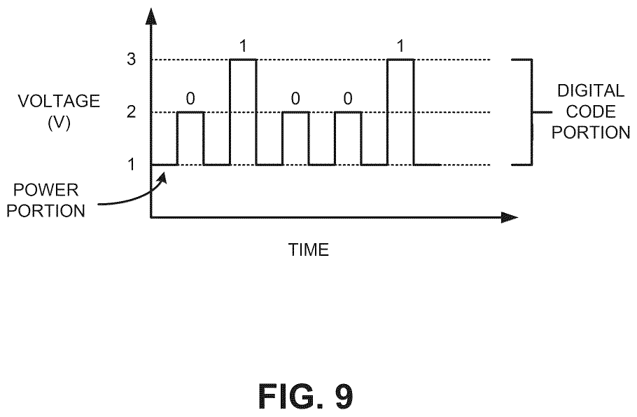

FIG. 9 shows a graph of an example waveform of the working signal described above in FIG. 8. The working signal is a voltage signal, sometimes referred to as the supply voltage or the supply line. The working signal can include both a power portion and at least one digital code portion. Digital communication can be achieved through modulation of the supply line over the power minimum. The supply voltage needs to be higher than a certain threshold to sustain power to the responders (e.g., the power portion) and can be further raised to different levels to carry digital codes (e.g., the at least one digital code portion). For example, the minimum power supply voltage is 1V. A fast rise and fall (e.g., a step for a time) in the supply voltage to 2V can signify a 0 bit and a fast rise and fall (e.g., a step for a time) in the supply voltage to 3V can signify a 1 bit. Using the 0 and 1 bits, the controller can send digital information to the responders. Each responder can regulate the responder's own local supply (e.g., with local references and/or a buffer) and can monitor the supply line to demodulate codes transmitted codes. By combining the digital data with the power signal, at least two shared connections that would traditionally need to be used can be eliminated.

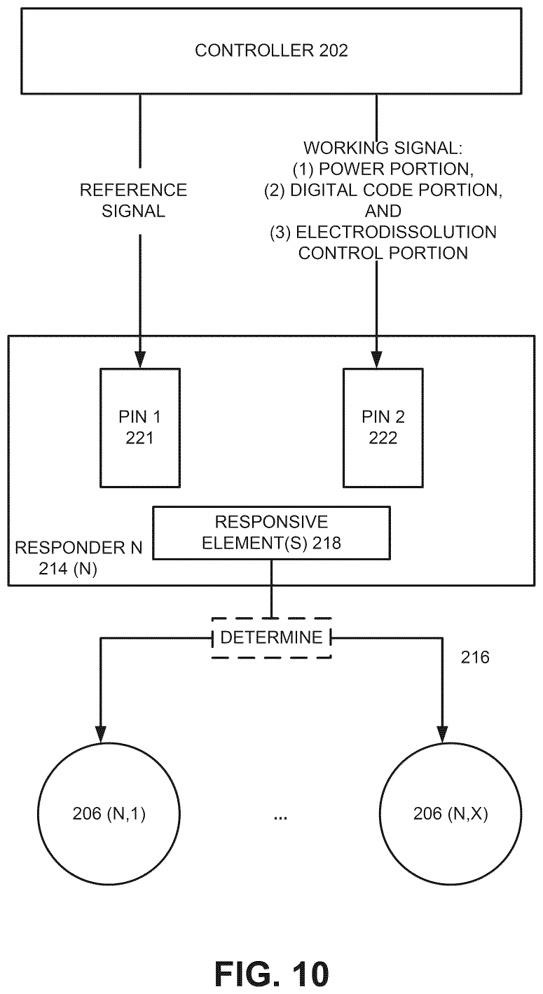

FIG. 10 shows an example diagram of the controller responder architecture with the two-line control line bundle of FIG. 8, element B. The controller-responder architecture with the two-line control line bundle can include a controller 202 and a plurality of responders (represented as Responder N 214 (N)) having two pins. In this example, the electrical signal transmitted from the controller 202 to each of the plurality of responders (example shown as Responder 214 (N)) can include a ground and/or reference signal and a working signal. It should be understood that the pin numbering is for ease of description and any pin could connect with any line. Pin 1 221 can connect the reference signal (and/or ground signal) line from the controller 202 to the responder(s). The ground and/or reference signal line can send the ground and/or reference signal to each of the plurality of responders. Pin 2 222 can connect a signaling line from the controller 202 to the responder(s) to send the working signal from the controller to the responder(s). The signaling line can transmit the working signal to each of the plurality of responders. The working signal is the compound signal. In the two-line control line bundle example the working signal can include a power signal portion for powering the responder(s), at least one digital code portion overlayed over the power signal to indicate at least the timing and selection of a responder and an associated drug reservoir to release a drug, and an electrodissolution signal portion overlayed over the power signal to trigger electrodissolution of the electrode of the selected drug reservoir.

For example, the at least one digital code portion can select at least one the plurality of responders (e.g., Responder 214 (N)) and then select at least one electrode (e.g., from electrodes 206 (N,1)-(N,X)) connected to the selected at least one of the plurality of responders to be electrodissolved. The electrodissolution signal portion can be sent to the selected at least one electrode through the selected at least one of the plurality of responders to trigger electrodissolution of the selected at least one electrode. The electrodissolution signal portion can be, for example, a voltage signal to be at least partially sent through a responder to the electrode covering the selected drug reservoir. The responder(s) can also include at least one responsive element 218 such as a chip, a memory, or a processor that can react to receiving at least a part of an electrical signal from the controller 202. As shown in FIG. 8, the reaction of the responder can be to determine which of the electrodes) to electrodissolve to release the drug from the selected drug reservoir. When the determination is made (based on the at least one digital code portion) at least a portion of the electrodissolution signal portion can be sent from the responder (e.g., Responder 214 (N)) to the determined electrode of electrodes (e.g., one of electrode 206 (N,1)-(N,X)).

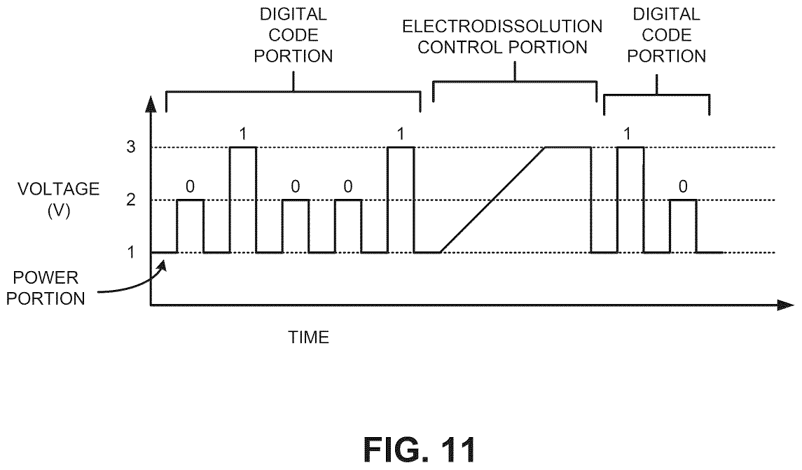

FIG. 11 shows a graph of an example waveform of the working signal described above with respect to FIG. 10. The working signal is a voltage signal, sometimes referred to as the supply voltage or the supply line. The working signal can include a power portion, at least one digital code portion, and an electrodissolution control portion. The digital communication achieved through modulation of the supply line as described with respect to FIG. 9 can be extended to include analog signaling as well. The supply voltage can change between a lower limit determined by the minimum power supply requirements (e.g., 1 V) (e.g., the power portion), and an upper limit determined by device reliability thresholds. Digital code can be communicated, for example with predetermined patterns of 0 and 1 bits between the upper and lower limits. For example, a fast rise and fall (e.g., a step for a time) in the supply voltage to 2V can signify a 0 bit and a fast rise and fall (e.g., a step for a time) in the supply voltage to 3V can signify a 1 bit. An analog signal can also be modulated into the supply voltage in between the upper limit and the lower limit. The analog signal can be chronologically after at least one digital code portion selecting the responder. For example, the ramp signal can be an electrodissolution control waveform that can be added on top of the minimum power portion. To derive the actual electrodissolution voltage to be sent to the selected electrode, the responder associated with the selected electrode can subtract the power portion (e.g., 1 V) from the supply voltage (e.g., through an analog voltage subtractor circuit). By carrying both digital and analog signals on top of the power portion of the supply line the controller can share only two connections (signaling line and ground/reference line) to the plurality of responders (e.g., two to each or two to all depending on the configuration). The plurality of responders in the controller-responder architecture described herein with the two- and three-line control line bundles can include non-volatile memory for singulation and analog blocks, such as voltage regulators, demodulators, and voltage subtractor circuits to demodulate and use the modulated supply line (e.g., working signal).

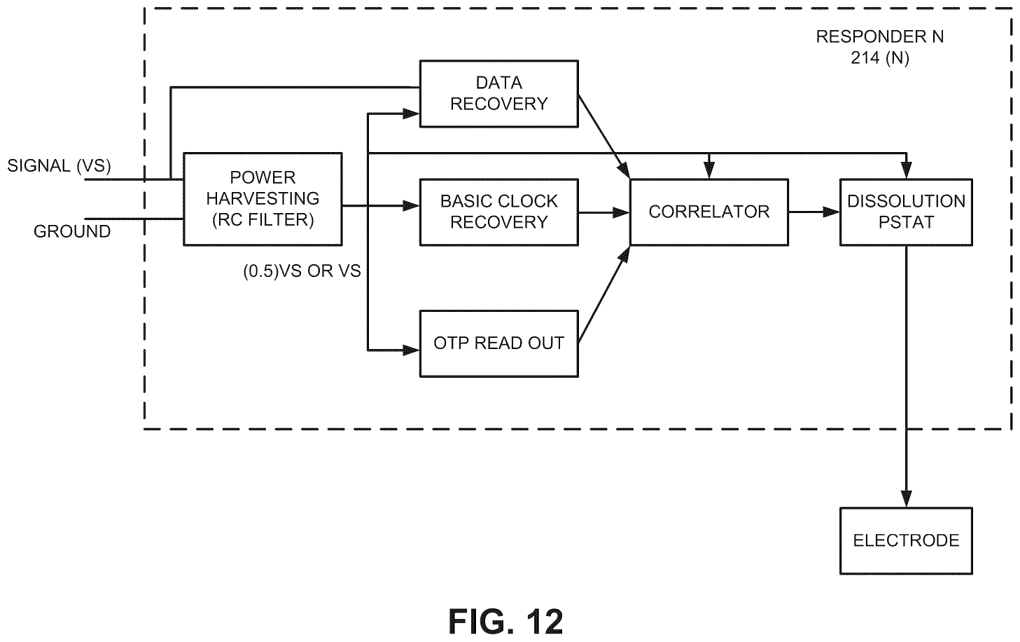

Referring now to FIG. 12, a block diagram of example internal elements of responder 214 (N) is shown for a two pin responder (where only a working signal and a ground signal are input). The controller 202 (not shown) can send the signal (e.g., working signal) and the ground (e.g., ground and/or reference signal) to the responder 214 (N). The working signal can be Manchester encoded to guarantee at least 50% average signal) for power harvesting by the responder 214 (N). The digital code portion of the working signal can have enough bits to avoid redundancy and allow selectability for the number of the plurality of drug reservoirs. For example, 31 drug reservoirs can be covered by 5 bits of code, but more tunability would be possible with more bits, such as 10 bits. Each of the responders 214 (N) can be programmed with a unique ID (e.g., during manufacturing at the wafer probe level or at the post module assembly stage) with a one-time programmable memory (OTP). For example, the unique ID can be burned into a memory of each of the responders 214 (N) during the manufacturing and/or assembly processes. In this way, the data on the controller and the responders 214 (N) can be in sync and the responders can be addressable by the controller.

In the responder 214 (N) a power harvesting component, such as an RC filter, can receive at least a portion of the working signal (e.g., at least the power portion) and the ground signal and harvest the power portion of the working signal to power the responder. A portion of the ground signal can be passed from the power harvesting component through to all the other components of the responder 214 (N). The responder 214 (N) can also include a data recovery component, a basic clock recovery component, and an OTP readout component configured to read and react to at least one portion of the working signal. The data recovery component can receive at least a portion of the working signal directly from the controller and a portion of the working signal that has already been harvested by the power harvesting component. The data recovery portion can demodulate at least a portion of the digital code included in the working signal. The basic clock recovery component can receive at least the portion of the working signal including at least the clock portion of the digital code signal portion, which the clock recovery component can demodulate. The OTP read out component can receive a portion of the working signal from power harvesting component and can demodulate the unique ID included in the working signal, which can be used to determine if the responder 214 (N) is the responder associated with the selected drug reservoir, for example. The outputs from each of the data recover component, basic clock recovery component, and OTP read out can be sent to the correlator component (along with a portion of the working signal that has passed through the power harvesting component) and then to the Dissolution PSTAT component (also along with a portion of the working signal that has passed through the power harvesting component). The output of the Dissolution PSTAT component can then be sent to the electrode when the working signal signaled that that electrode was the one to be triggered for electrodissolution.

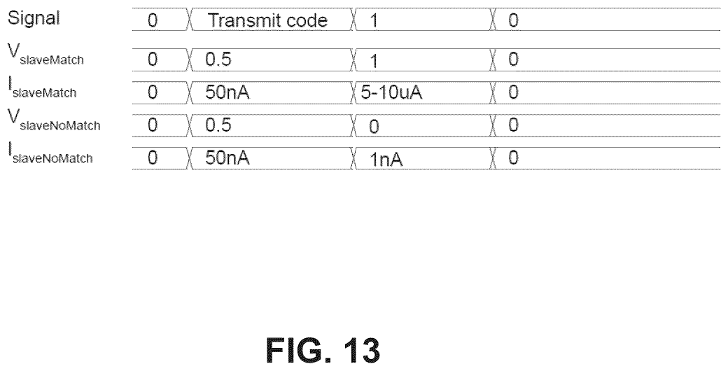

FIG. 13 shows an example table for communicating and demodulating the working signal of the controller-responder architecture having two or three-line control line bundles (wherein the difference is that the three-line control line bundle can transmit a fully separate electrodissolution control signal). When a particular drug reservoir needs to be released, the controller can send an electrical signal (e.g., the working signal and the reference and/or ground signal) along the control line bundle that addresses that particular drug reservoir. When the responders begin to toggle, all of the responders wake up and begin decoding the electrical signal (e.g., the Manchester encoded signal) in a low power operating mode (e.g., about 50 nA for each drug reservoir). When the responder that corresponds to the code included in the electrical signal picks up the correct key (e.g., the code matches the unique ID of the responder), then that responder can go into a high power harvesting mode (e.g., the signal drives to DC VDD voltage) and can drive the dissolution PSTAT for as long as the signal line is held high at VDD to send the electrodissolution triggering signal (e.g., voltage waveform) to the electrode of the particular drug reservoir. At the same time, each of the other responders that did not match to the correct key can go into a deep sleep state (powered at about 1 nA) for as long as there remains an electrical signal on the control line bundle to allow a majority of the harvestable energy on the signal line to go towards the particular drug reservoir. When the electrical signal goes back to zero (e.g., with no transitions), then all of the responders can rest their operating mode, ready for the next transmit code from the controller. Additional digital code signals with other instructions can be sent along the electrical signal as well, for example, check whether the drug has been released from the particular drug reservoir, check impedance, etc. Another connection line (e.g., a third ring in FIG. 4) can be added for additional controller-responder communication or a global protocol can be designed for the responders to communicate bi-directionally on the same signal line that is used to transmit power and instructions from the controller.

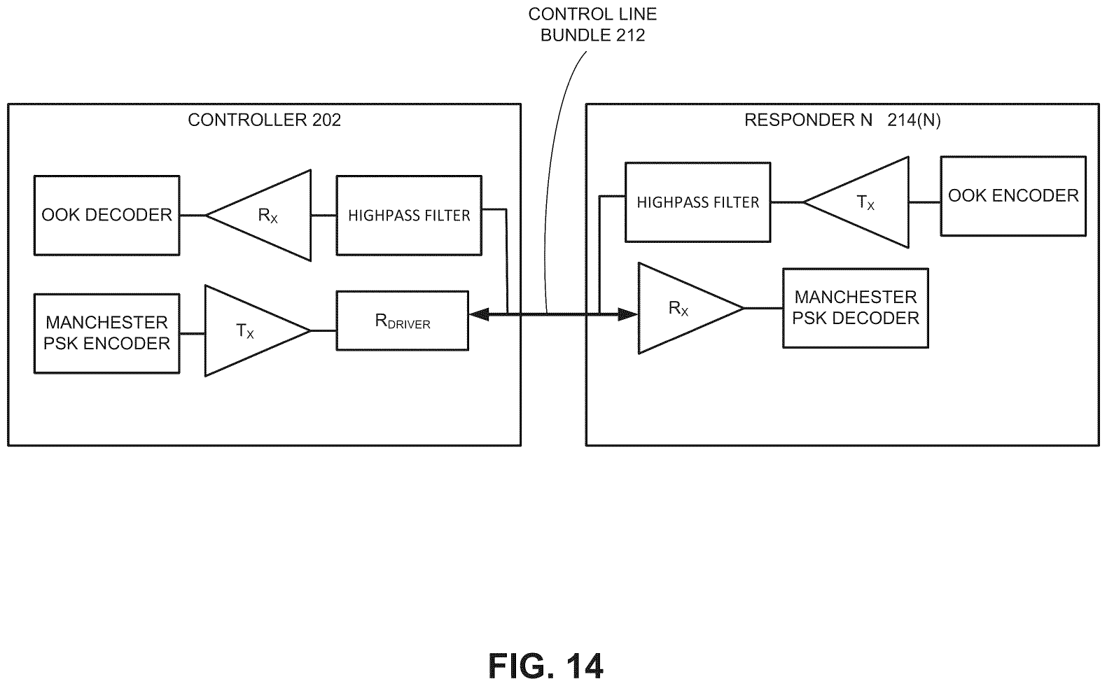

FIG. 14 shows an example of two way communication between controller 202 and a responder 214 (N) connected by a control line bundle 212. Nominal signal and power from the controller 202 to the responder 214 (N) can be done with Manchester PSK encoding. The controller 202 can include, for example, a Manchester pre-shared key (PSK) encoder that feeds the electrical signal to a transmitter (Tx) then to a driver (R_{driver}) to the control line bundle. The responder 214 (N) can include a receiver (Rx) and a Manchester PSK decoder to decode the electrical signal. Responder to controller communication can be done in a high frequency domain with OOK encoding, for example. The responder 214 (N) can include an OOK encoder that feeds a signal to a transceiver (Tx), which feeds to a high pass filter and sends back along the control line bundle 212. The controller 202 can also include a high pass filter connected to the control line bundle 212 that feeds the high frequency OOK signal to a receiver (Rx) and then to an OOK decoder. It should be understood that this is only one example of two-way communication between the controller 202 and the plurality of responders 214 (N) on the same signal wire (e.g., control line bundle 212), and that others are contemplated.

### IV. Method

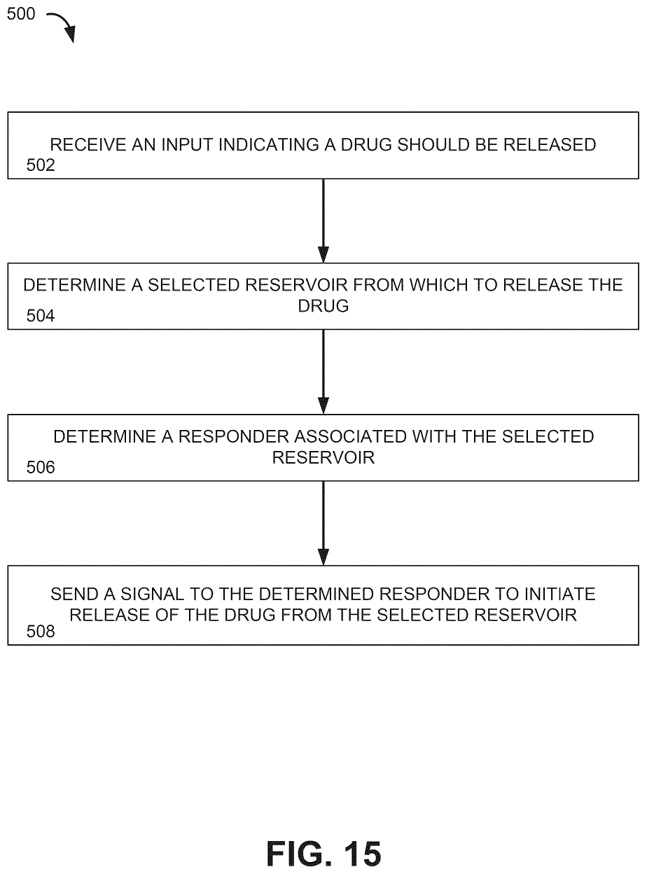

Another aspect of the present disclosure can include example methods 500, 600, 700, 800, and 900 (shown in FIGS. 15, 16, 17, 18, and 19) for controlling an active drug dispensing ophthalmic device with a compound electrical signal using an ophthalmic device with a controller-responder architecture (examples of the ophthalmic device and the architecture are shown in FIGS. 2-14). The methods 500, 600, 700, 800, and 900 are illustrated as process flow diagrams with flowchart illustrations that can be implemented as ophthalmic device 200 (or ophthalmic device 300, contact lens 350, ophthalmic device 400, or contact lens 450) as shown at least in FIGS. 2-6.

For purposes of simplicity, the methods 500, 600, 700, 800, and 900 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 500, 600, 700, 800, and 900. It should be noted that one or more steps of the methods 500, 600, 700, 800, and 900 can be executed by a hardware processor.

FIG. 15 shows an example method 500 for generally actively controlling the release of a drug from an ophthalmic device (e.g., ophthalmic device 200, 300, contact lens 350, 400, contact lens 450, or the like) having a plurality of drug reservoirs and a controller-responder architecture. The ophthalmic device can include the controller, a plurality of responders connected to the controller via at least one control line bundle, and the plurality of drug reservoirs, wherein each of the plurality of drug reservoirs is configured to hold a volume of the drug and has an opening covered by an electrode, wherein each of the plurality of responders is in electrical connection with the electrode covering at least one of the plurality of drug reservoirs. The drug can be actively released as part of a treatment plan for an ocular disorder based closed loop and/or open loop control principles. At 502, the controller of the ophthalmic device can receive an input indicating a drug should be released. The input can be a prescheduled input (e.g., time based), an input from a sensor(s) (e.g., based on one or more measured physiological parameters at a time), and/or a manual input (e.g., from the user or a medical professional associated with the user) via an external device (e.g., smartphone, computer, etc.) in communication with the controller. Depending on the complexity of the ophthalmic device the input can include, or can be used by the controller to determine, information on what type of drug to be released (e.g., if the ophthalmic device contains more than one type of drug), what reservoir(s) to release the drug from, or the like.

At 504, the controller of the ophthalmic device can determine a selected drug reservoir from a plurality of drug reservoirs from which to release the drug and at 506 a responder associated with the selected reservoir can be determined. Each of the drug reservoirs and responders can have an individual identifier. The controller can, for example, include a memory of drug reservoirs already opened and a predetermined schedule for which reservoirs to release at a time (e.g., in a patterned manner). In more complex control the controller can determine which reservoir to select based on the input. At 508, an electrical signal can be sent, by the controller, to the determined responder to initiate release of the drug from the selected drug reservoir. The electrical signal can be one or more voltage waveforms. The responder can send an electrodissolution signal to the electrode of the selected drug reservoir to begin electrodissolution of the electrode and trigger release of the drug.

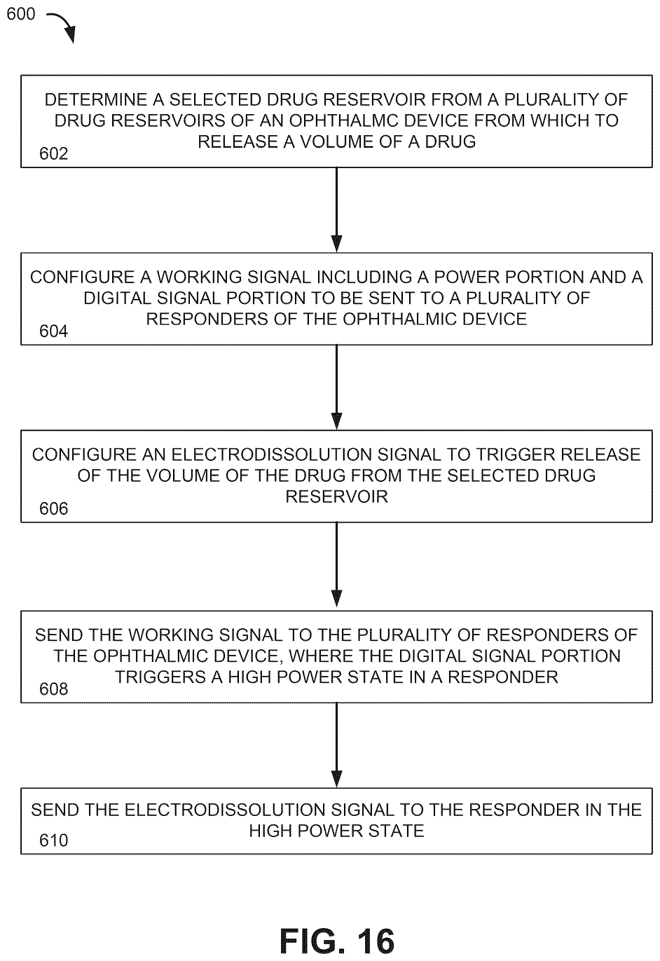

FIG. 16 shows an example method 600 for actively controlling release of a drug from an ophthalmic device (e.g., ophthalmic device 200, 300, contact lens 350, 400, contact lens 450, or the like) having a plurality of drug reservoirs and a controller-responder architecture with a three line control bundle and three pin responders (as described above with respect to FIG. 7, element A, FIG. 8 and FIG. 9). At 602, a selected drug reservoir from the plurality of drug reservoirs (and the associated responder) of the ophthalmic device from which to release a drug (e.g., a volume of the drug held in the drug reservoir) can be determined. The determination can be based on, for example, a predetermined dosage schedule (amount of drug and time), an input from a sensor, or a manual input (e.g., via a connected smartphone or computer or the like). Only a full drug reservoir can be selected (e.g., a drug reservoir still storing the drug and having an electrode cover). For instance, the controller can include a memory of which drug reservoirs have already been used or can be in two-way communication through the responder to the reservoirs to determine which are still in electrical communication (e.g., not electrical communication if not electrode remains).

At 604, a working signal can be configured to be sent to the plurality of responders of the ophthalmic device via the at least one control line bundle. The working signal can include a power portion and at least one digital signal (also referred to as a digital code signal). The power portion can be configured to power the plurality of responders to a low power mode. The power portion of the signal can include a base power voltage to power the plurality of responders. For example, the power portion of the signal can be a steady 1 V minimum for the entire time of the working signal. The digital signal portion can include digital information that can be decoded by the plurality of responders. For example, the digital signal portion can be keyed to a responder (e.g., include a two-bit digital code that matches with a unique identifier of the responder) in communication with the electrode covering the selected drug reservoir and can make the responder in communication with the electrode covering the selected drug reservoir enter a high power state and a remainder of the plurality of responders (which do not match the key) to enter a sleep state. The digital signal portion can also include, for example, a digital code for choosing the selected drug reservoir and clock information (e.g., when to trigger electrodissolution of the electrode of the selected drug reservoir). The digital signal portion of the working signal can include one or more predetermined voltage patterns layered on top of the base power voltage. For example, a step for a time period in a waveform of the working signal to a first voltage level (e.g., 2 V) over the base power voltage can signify a 0 bit and another step for another time period in the waveform of the working signal to a second voltage level (e.g., 3 v) over the base power voltage can signify a 1 bit. The 1 bit and 2 bit steps can be patterned together in a predetermined manner to send digital information to the responders.

At 606, an electrodissolution signal (also referred to as an electrodissolution control signal) can be configured, by the controller, to be sent to the responder in the high power state to trigger release of the volume of the drug stored in the selected drug reservoir. The electrodissolution signal can be a voltage waveform configured to trigger electrodissolution of an electrode. For example, an analog ramp starting at 2 V and ramping down to 0 V. The electrodissolution signal can be sent along a separate line of the control line bundle from the working signal and can be sent only to (or only received by) the responder in the high power state. Additionally, a reference and/or ground signal can be configured by the controller and can be sent to the plurality of responders via a reference line (which can be the third line of the control line bundle or could be physically separate from the control line bundle) at the same time as the working signal and the electrodissolution signal. At 608, the working signal can be sent, by the controller over the working signal line of the control line bundle, to the plurality of responders of the ophthalmic device and can trigger the high power state in the desired responder and the sleep state of the remainder. At 610, the electrodissolution signal can be sent to the responder in the high power state. In response to receiving the working signal and the electrodissolution signal (and the reference and/or ground signal) the responder in the high power state can then send the electrodissolution signal to the electrode covering the selected drug reservoir to trigger electrodissolution of the electrode and release of the drug stored therein.

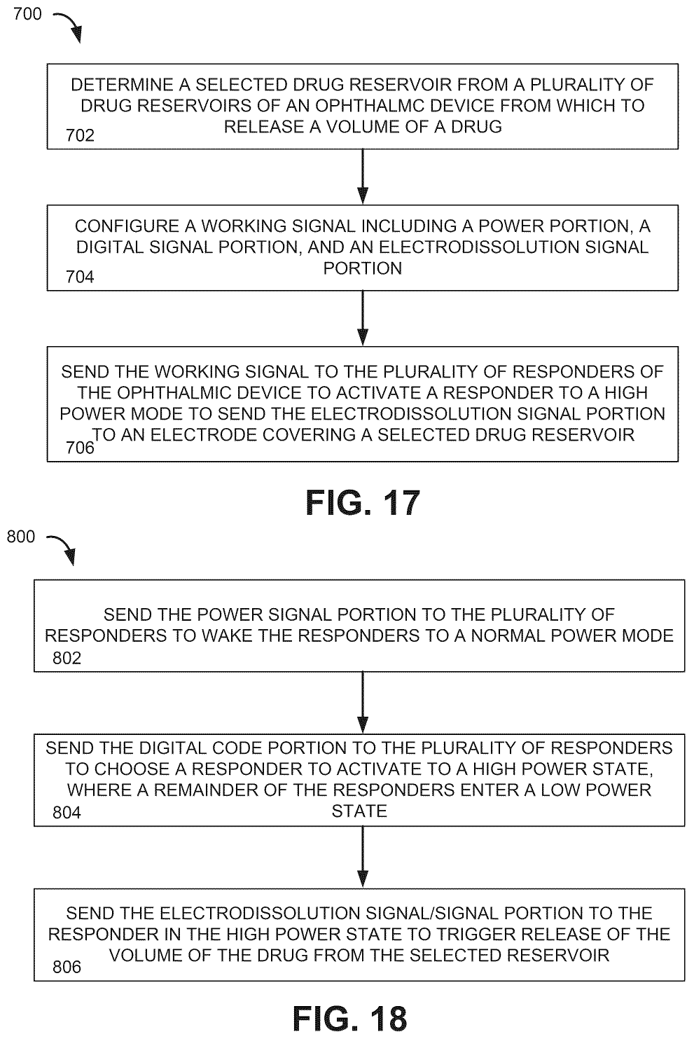

FIG. 17 shows an example method 700 for actively controlling release of a drug from an ophthalmic device (e.g., ophthalmic device 200, 300, contact lens 350, 400, contact lens 450 or the like) having a plurality of drug reservoirs and a controller-responder architecture with a two line control bundle and two pin responders (as described above with respect to FIG. 7, element B, FIG. 10 and FIG. 11). Similar to method 600, at 702 a selected drug reservoir from the plurality of drug reservoirs (and the associated responder) of the ophthalmic device from which to release a drug (e.g., a volume of the drug held in the drug reservoir) can be determined. The determination can be based on, for example, a predetermined dosage schedule (amount of drug and time), an input from a sensor, or a manual input (e.g., via a connected smartphone or computer or the like). Only a full drug reservoir can be selected (e.g., a drug reservoir still storing the drug and having an electrode cover). For instance, the controller can include a memory of which drug reservoirs have already been used or can be in two-way communication through the responder to the reservoirs to determine which are still in electrical communication (e.g., not electrical communication if not electrode remains).

At 704, a working signal can be configured to be sent to the plurality of responders of the ophthalmic device via the at least one control line bundle. The working signal can include a power portion, at least one digital signal (also referred to as a digital code signal), and an electrodissolution signal portion (also referred to as an electrodissolution control signal portion). The power portion can be configured to power the plurality of responders to a low power mode. The power portion of the signal can include a base power voltage to power the plurality of responders. For example, the power portion of the signal can be a steady 1 V minimum for the entire time of the working signal. The digital signal portion can include digital information that can be decoded by the plurality of responders. For example, the digital signal portion can be keyed to a responder (e.g., include a two-bit digital code that matches with a unique identifier of the responder) in communication with the electrode covering the selected drug reservoir and can make the responder in communication with the electrode covering the selected drug reservoir enter a high power state and a remainder of the plurality of responders (which do not match the key) to enter a sleep state. The digital signal portion can also include, for example, a digital code for choosing the selected drug reservoir and clock information (e.g., when to trigger electrodissolution of the electrode of the selected drug reservoir). The digital signal portion of the working signal can include one or more predetermined voltage patterns layered on top of the base power voltage. For example, a step for a time period in a waveform of the working signal to a first voltage level (e.g., 2 V) over the base power voltage can signify a 0 bit and another step for another time period in the waveform of the working signal to a second voltage level (e.g., 3 v) over the base power voltage can signify a 1 bit. The 1 bit and 2 bit steps can be patterned together in a predetermined manner to send digital information to the responders. The electrodissolution signal portion can be configured to be sent to (or to only be received by) the responder in the high power state and to trigger release of the volume of the drug stored in the selected drug reservoir. Where the responder can take the electrodissolution signal portion and at least partially send it to the electrode to begin electrodissolution of the electrode. The electrodissolution signal portion can be configured into the working signal chronologically after the digital signal portion. The electrodissolution signal portion can be an analog waveform signal layered over the power portion of the signal, such as a ramp. For example, the analog ramp can start at 3V (2 V plus the 1 V minimum power) and ramp down to 1 V (the minimum power voltage).

At 706, the working signal, including the power portion, at least one digital signal portion, and the electrodissolution signal can be sent to the plurality of responders via a single working signal line of the at least one control line bundle. Additionally, a reference and/or ground signal can be configured by the controller and can be sent to the plurality of responders via a reference line (which can be the second line of the control line bundle or could be physically separate from the control line bundle) at the same time as the working signal. Sending the working signal can activate a determined responder to a high power mode and send the remainder of responders to a sleep state (via the power portion and the digital code portion). In response to receiving the electrodissolution signal portion and power portion of the working signal the responder in the high power state is configured to sends the electrodissolution signal portion to the electrode covering the selected drug reservoir to trigger electrodissolution of the electrode.

FIG. 18 shows an example method 800 for sending the working signal (in an ophthalmic device having a controller-responder architecture with a two line control line bundle or the working signal and the electrodissolution signal (in an ophthalmic device having a controller-responder architecture with a three control line bundle) for triggering electrodissolution of an electrode covering a drug reservoir. At 802, the power signal portion (e.g., a 1 V steady, or minimum, voltage waveform) of the working signal can be sent to the plurality of responders to wake the plurality of responders from a low power and/or sleep state to a normal power mode. Then, while the power signal portion is still being sent, the digital code portion can be sent (804) over the power signal portion (e.g., 2 V steps for a time and 3 V steps for a time in patterns, with a 1 V minimum between each step) to choose a responder to activate to a high power state and to send the remainder to (or back to) a sleep state. The digital code can be keyed to a unique identifier of the responder to active to the high power mode, and the selected drug reservoir associated with the responder. The digital code portion can also include, for example, information about timing for triggering the release of the drug or the like. At 806, the electrodissolution signal portion can be sent, over the power portion of the signal still being sent, but after transmission of the digital code choosing the responder, to the responder in the high power state to trigger release of the drug from the selected reservoir. The electrodissolution signal may be sent to all the plurality of responders but only received by the responder in the high power state. Alternatively, the electrodissolution signal can be sent simultaneously with the working signal only sending a power portion, after the digital code portion has been transmitted to trigger release of the drug from the selected reservoir.

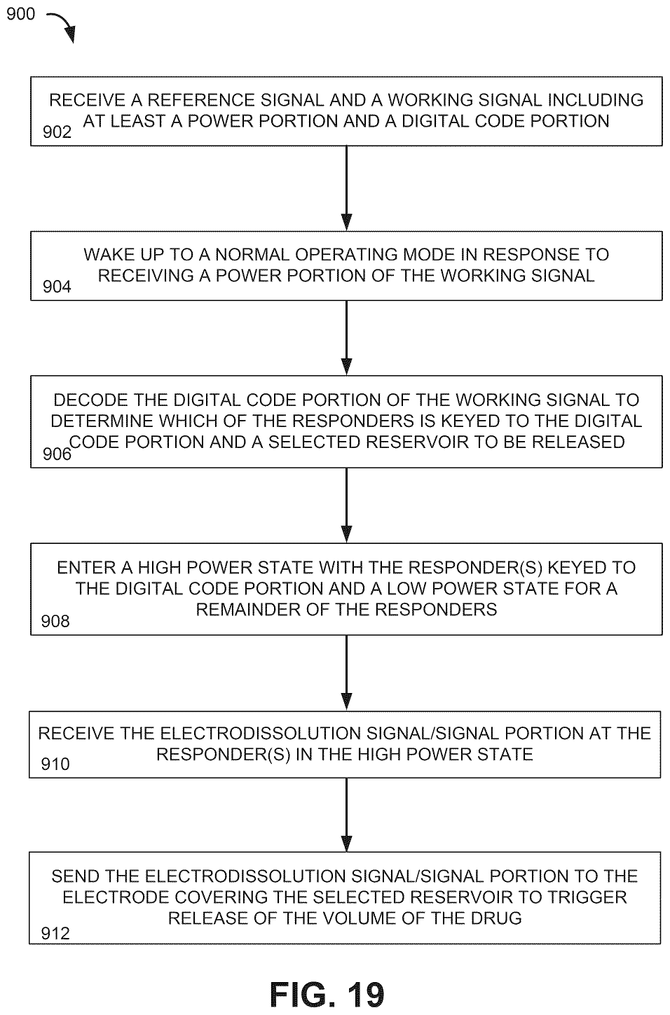

FIG. 19 shows an example method 900 of how the responders react to the electrical signals from the controller to actively control drug release from a selected drug reservoir. At 902, the plurality of responders can each receive a reference and/or ground signal and a working signal. The working signal can include at least a power portion and at least one digital code portion. The working signal may include, after the at least one digital code portion, an electrodissolution signal portion or a separate electrodissolution signal may be received after the working signal has been received. At 904, the plurality of responders can each wake up to a normal operating mode in response to receiving the power portion of the working signal. At 906, the plurality of responders can each decode the digital code portion of the working signal to determine which of the plurality of responders is keyed to a code in the digital code portion and which of the connected plurality of drug reservoirs is the selected drug reservoir to be released. This can be done via an OTP unique identifier. The responder (or responders) keyed to the digital code can, at 908, enter a high power state and the remainder of the responders of the plurality of responders can enter a low power and/or sleep state where they cannot respond until a new initial power portion (e.g., going from 0 to 1 V) of a signal is received. Then at 910, the responder(s) in the high power state can receive the electrodissolution signal portion over the power portion and/or the working signal with only the power portion and a separate electrodissolution signal. At 912, the responder(s) in the high power state can then send the electrodissolution signal or the electrodissolution signal portion to the electrode covering the selected drug reservoir to trigger release of the drug from that drug reservoir by starting electrodissolution of the electrode.

### V. Examples

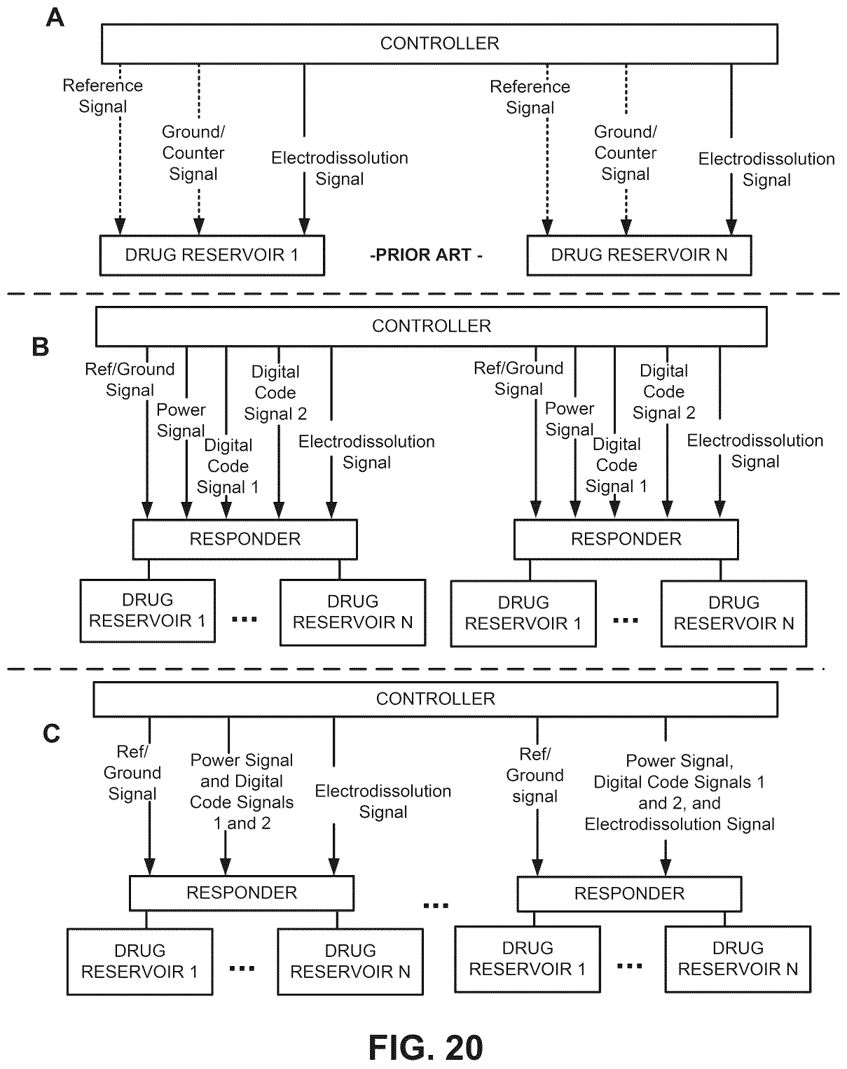

FIG. 20 shows illustrations comparing different control architectures for active drug dispensing ophthalmic devices. More specifically, the example shown in FIG. 20, element A, is a traditional controller-reservoir architecture; while the example shown in FIG. 20, element B, is a controller-responder architecture with normal signaling, which is an improvement compared to FIG. 20, element A; and FIG. 20, element C, illustrates controller-responder architectures with three line and two line compound signaling, which are both improvements to both of FIG. 20, elements A and B.

As noted, FIG. 20, element A, represents a traditional controller-reservoir architecture used in ophthalmic devices for active drug dispensing. Each of the drug reservoirs (represented as Drug Reservoir 1-Drug Reservoir N) is separately and independently connected to the controller. For each of the drug reservoirs, at least an electrodissolution signal must be sent to a drug reservoir to actively control the release of drugs (e.g., to choose when the drug is dispensed and from what reservoir). Additionally, a reference voltage signal and a counter and/or ground signal are needed to activate the reservoir but are sent to not-dissolvable electrodes associated with the drug reservoir. These signals are needed for each instance of drug release. As the number of drug reservoirs using this control architecture increases the number of connections proportionally increases, and as the number of connections increases the required size of the controller increases to accommodate the connections. Additionally, each of the growing number of connections requires enough room to be insulated separately from each other. The overall size of the ophthalmic devices available for these components is limited to what can comfortably fit on and/or in the eye and not harm the user's vision. All of which exponentially increases the difficulty of manufacturing ophthalmic devices as the number of drug reservoirs included therein increases past a certain point.

As further noted, FIG. 20, element B, represents a use of a controller-responder architecture for active dispensing of a drug from an ophthalmic device (as shown in FIG. 2) with a traditional form of control signaling. In this example, the controller is in communication with any number of responders (two shown for representative purposes only) and each responder is in communication with at least one, but any number greater than one, drug reservoirs (represented as Drug Reservoir 1-Drug Reservoir N, with two shown for representative purposes only). As the number of drug reservoirs increases, the drug reservoirs can be connected to a responder with a simpler connection while a more complex connection can connect the controller to the responders. For each of the responders at least five signals are necessary to actively control the release of drugs (e.g., to choose when the drug is dispensed and from what reservoir): a reference and/or ground signal, a power signal, a first digital signal (e.g., a clock signal), a second digital signal (e.g., a reservoir selection signal), and an electrodissolution signal. From each responder, the requisite parts of these signals can be sent to a selected drug reservoir to at least actively electrodissolve the electrode covering the selected drug reservoir. In this architecture the connections to the reservoir are simplified but may still pose difficulties with greater numbers of responders and drug reservoirs due to the complexities of the connections from the controller to the responders.

As additionally noted, FIG. 20, element C, represents controller-responder architecture for active drug dispensing from an ophthalmic device (as shown in FIG. 2) with three line (left-side) and/or two line (right-side) compound signaling, as discussed in detail above with regard to FIGS. 7-11, for example. These architectures with the compound signaling improve upon the design of FIG. 20, element B, through the additional use of compound signals sharing one line. As shown on the left, the five separate signals required for the controller to control one responder can be reduced to three through the combination of the power signal and the digital code signals (as described above in greater detail). As shown on the right, the five separate signals required for the controller to control one responder can be further reduced to three through the combination of the electrodissolution signal with the power and digital code signals. Additional space is saved, and ease of manufacturability is increased, as the complexity of the connections from the controller to the responders is decreased. However, there may be design and/or computational reasons for choosing to include or not include the electrodissolution signal with the power signal and the digital code signal.

From the above description, those skilled in the art will perceive improvements, changes, and modifications. Such improvements, changes and modifications are within the skill of one in the art and are intended to be covered by the appended claims.

1. An ophthalmic device comprising:
a controller configured to send an electrical signal to control at least one of a plurality of drug reservoirs;
at least one control line bundle configured to transmit the electrical signal to a plurality of responders;
the plurality of responders, each connected to the controller via the at least one control line bundle, wherein each of the plurality of responders is configured to receive the electrical signal; and
the plurality of drug reservoirs, wherein each of the plurality of drug reservoirs is configured to hold a volume of a drug and is covered by an electrode,
wherein each of the plurality of responders is in electrical communication with the electrode of at least one of the plurality of drug reservoirs.

2. The ophthalmic device of claim 1, wherein the electrical signal comprises a reference signal and a working signal and wherein each of the at least one control line bundle comprises:
a ground line configured to transmit the reference signal to each of the plurality of responders; and
a signaling line configured to transmit the working signal to each of the plurality of responders, wherein the working signal comprises:
a power signal portion,
at least one digital code signal portion configured to select at least one the plurality of responders and then select at least one electrode connected to the selected at least one of the plurality of responders be electrodissolved, and
an electrodissolution signal portion configured to be received by the selected at least one of the plurality of responders and then sent to the selected at least one electrode to trigger electrodissolution of the selected at least one electrode.

3. The ophthalmic device of claim 1, wherein the electrical signal comprises a reference signal, a working signal, and an electrodissolution signal wherein each of the at least one control line bundle comprises:
a ground line configured to send the reference signal to each of the plurality of responders;
a signaling line configured to transmit the working signal to each of the plurality of responders; and
an electrodissolution control line configured to send the electrodissolution signal to the selected at least one electrode through the selected at least one of the plurality of responders to trigger electrodissolution of the selected at least one electrode.

4. The ophthalmic device of claim 1, wherein the at least one control line bundle comprises a plurality of control line bundles, wherein one of the plurality of control line bundles is paired with one of the plurality of responders.

5. The ophthalmic device of claim 1, wherein each of the plurality of responders comprises 2 pins or 3 pins to receive the at least one control line bundle.

6. The ophthalmic device of claim 1, wherein the controller is configured to receive feedback signals from at least one of the plurality of responders through the at least one control line bundle.

7. The ophthalmic device of claim 1, wherein the controller comprises a microprocessor and each of the plurality of responders comprises at least a portion of another microprocessor.

8. The ophthalmic device of claim 1, wherein each of the plurality of responders are associated with a unique ID.

9. The ophthalmic device of claim 2, wherein each of the responders further comprises at least one sensor configured to detect at least one characteristic of the electrodissolution and/or a drug stored in the at least one of the plurality of drug reservoirs.

10. The ophthalmic device of claim 1, wherein the controller and the at least one control line bundle are embodied in an electronics module and the plurality of responders and the plurality of drug reservoirs are embodied in at least one drug container module.

11. A method comprising:
determining, by a controller of an ophthalmic device, a selected drug reservoir from a plurality of drug reservoirs from which to release a drug, wherein the ophthalmic device further comprises:
a plurality of responders connected to the controller via at least one control line bundle, and
the plurality of drug reservoirs, wherein each of the plurality of drug reservoirs is configured to hold a volume of the drug and has an opening covered by an electrode,
wherein each of the plurality of responders is in electrical connection with the electrode covering at least one of the plurality of drug reservoirs;
configuring, by the controller, a working signal comprising:
a power portion configured to power the plurality of responders to a low power mode, and
a digital signal portion keyed to a responder in communication with the electrode covering the selected drug reservoir and configured to make the responder in communication with the electrode covering the selected drug reservoir enter a high power state and a remainder of the plurality of responders enter a sleep state;
configuring, by the controller, an electrodissolution signal to be sent to the responder in the high power state to trigger release of the volume of the drug stored in the selected drug reservoir; and
sending, by the controller, the working signal and then the electrodissolution signal to the plurality of responders via the at least one control line bundle,
wherein in response to receiving the working signal and the electrodissolution signal the responder in the high power state is configured to send the electrodissolution signal to the electrode covering the selected drug reservoir to trigger electrodissolution of the electrode.

12. The method of claim 11, further comprising sending, by the controller, a reference signal to the plurality of responders via a reference line at a same time as the working signal and the electrodissolution signal.

13. The method of claim 11, wherein the power portion comprises a base power voltage to power the plurality of responders.

14. The method of claim 13, wherein the digital signal portion comprises a predetermined voltage pattern layered on top of the base power voltage.

15. The method of claim 14, wherein a step for a time period in a waveform of the working signal to a first voltage level over the base power voltage signifies a 0 bit and another step for another time period in the waveform of the working signal to a second voltage level over the base power voltage signifies a 1 bit.

16. A method comprising:
determining, by a controller of an ophthalmic device, a selected drug reservoir from a plurality of drug reservoirs from which to release a drug, wherein the ophthalmic device comprises:
a plurality of responders connected to the controller via at least one control line bundle, and
the plurality of drug reservoirs, wherein each of the plurality of drug reservoirs is configured to hold a volume of the drug and has an opening covered by an electrode,
wherein each of the plurality of responders is in electrical connection with the electrode covering at least one of the plurality of drug reservoirs;
configuring, by the controller, a working signal comprising:
a power portion configured to power the plurality of responders to a low power mode,
a digital signal portion keyed to a responder in communication with the electrode covering the selected drug reservoir and configured to make the responder in communication with the electrode covering the selected drug reservoir enter a high power state and a remainder of the plurality of responders enter a sleep state, and
an electrodissolution signal portion to be sent to the responder in the high power state to trigger release of the volume of the drug stored in the selected drug reservoir; and
sending, by the controller, the working signal to the plurality of responders via the at least one control line bundle,
wherein in response to receiving the working signal the responder in the high power state is configured to send the electrodissolution signal portion to the electrode covering the selected drug reservoir to trigger electrodissolution of the electrode.

17. The method of claim 16, further comprising sending, by the controller, a reference signal to the plurality of responders via a reference line at a same time as the working signal.

18. The method of claim 16, wherein the power portion comprises a base power voltage for power the plurality of responders.

19. The method of claim 18, wherein the digital signal portion comprises a predetermined voltage pattern layered over the base power voltage or over the power portion of the signal.

20. The method of claim 19, wherein a step for a time period in a waveform of the working signal to a first voltage level over the base power voltage signifies a 0 bit and another step for another time period in the working signal to a second voltage level over the base power voltage signifies a 1 bit.

21. The method of claim 16, wherein the electrodissolution signal portion comprises an analog waveform signal layered over the power portion.

22. The method of claim 21, wherein the electrodissolution signal portion comprises a ramp waveform.
